(19) United States
(12) Patent Application Publication
(10) Pub. No.: US 2026/0301336 A1
(43) Pub. Date: Oct. 1, 2026

(54) AUGMENTED REALITY SENSORIMOTOR TRAINING METHOD

(71) Applicant: Neuro-Mod Inc., Oakville (CA)

(72) Inventors: Aimee Nelson, Stoney Creek (CA); Stevie Foglia, Oakville (CA); Gurman Johal, Brampton (CA); Zhen Gao, Hamilton (CA)

(21) Appl. No.: 19/629,681

(22) Filed: Mar. 26, 2026

Related U.S. Application Data
(60) Provisional application No. 63/778,086, filed on Mar. 26, 2025.

(51) Int. Cl. G06T 19/00; A61B 5/11; A63B 24/00

(52) U.S. Cl. CPC G06T 19/006; A61B 5/1114; A61B 5/1118; A63B 24/0006; A63B 24/0021; A63B 2024/0025

(57) ABSTRACT

A training system and computer-implemented method for determining spatial positions of training targets for visual trials presented within an augmented reality environment are disclosed. The system includes a processor, a display system configured to generate and present the visual trials, and one or more sensors configured to track movement of a user. A visual trial having at least one training target at a spatial position within the augmented reality environment is presented to the user. A range of motion of the user is assessed by evaluating alignment between the user's movement and the spatial position of the training target to determine training parameters for a subsequent visual trial. The subsequent visual trial is generated and presented, and the training parameters are applied to determine a subsequent spatial position of a subsequent training target within the augmented reality environment.

This application claims the benefit of U.S. Provisional Patent Application No. 63/778,086 filed on Mar. 26, 2025, entitled “AUGMENTED REALITY SENSORIMOTOR TRAINING METHOD”. The entirety of U.S. Provisional Patent Application No. 63/778,086 is incorporated herein by reference.

### FIELD

The present disclosure generally relates to augmented reality-based visual training, and in particular, to methods and systems for determining spatial positions of training targets for visual trials presented within an augmented reality environment.

### INTRODUCTION

The following is not an admission that anything discussed below is part of the prior art or part of the common general knowledge of a person skilled in the art.

Augmented reality technologies have been used in a variety of health, rehabilitation, exercise, and training contexts to present digital content within a user's real-world environment. By overlaying visual information onto a user's surroundings, such systems can provide interactive experiences that support guided activity in a manner that is natural and intuitive for the user.

Existing systems include various platforms and techniques for presenting visual guidance, facilitating therapeutic or exercise-related activities, and supporting user engagement. These technologies may be implemented in connection with wearable display devices, mobile computing platforms, or other computerized systems, and may be configured for use in clinical, home, wellness, sports, or other training environments.

### SUMMARY

The following introduction is provided to introduce the reader to the more detailed discussion to follow. The introduction is not intended to limit or define any claimed or as yet unclaimed invention. One or more inventions may reside in any combination or sub-combination of the elements or process steps disclosed in any part of this document including its claims and figures.

In one broad aspect, in accordance with some embodiments, there is generally provided a training system for determining spatial positions of training targets for visual trials presented within an augmented reality environment; the training system including a processor, a display system configured to present the visual trials within the augmented reality environment, and one or more sensors configured to track movement of a user; wherein the processor is configured to generate a visual trial presented to the user via the display system, the visual trial having at least one training target presented at a spatial position within the augmented reality environment; obtain the movement of the user from the one or more sensors during the visual trial; assess a range of motion of the user by evaluating an alignment between the movement of the user and the spatial position of the training target during the visual trial to determine training parameters for a subsequent visual trial defining a subsequent spatial position for a subsequent training target; generate the subsequent visual trial presented to the user via the display system; and apply the training parameters to determine the subsequent spatial position of the subsequent training target within the augmented reality environment.

In some embodiments, the processor is configured to determine the spatial position for the subsequent training target by selecting a start position and an end position for the subsequent training target from within range of motion limits of the user.

In some embodiments, the processor is configured to generate a trajectory for the training target between the start position and the end position along a path generated from a combination of straight angular components and sinusoidally varying angular components

In some embodiments, to assess the range of motion of the user, the processor is configured to determine whether movement of the user is substantially aligned with the training target for a predetermined threshold time.

In some embodiments, the processor is configured to determine the training parameters for the subsequent visual trial based on tracked movement associated with the visual trial; the tracked movement defining one or more performance metrics.

In some embodiments, the performance metrics include a smoothness metric for the visual trial based on the movement; the smoothness metric corresponding to the range of motion assessed over a predetermined period of time.

In some embodiments, the performance metrics include a success indicator associated with the visual trial; the success indicator indicating whether the user successfully tracked the training target for at least a threshold hold time while the training target was moving.

In some embodiments, to determine the training parameters for the subsequent visual trial, the processor is configured to evaluate the one or more performance metrics to determine a trial performance; and adjust a training parameter of the subsequent training target in the subsequent visual trial based on the trial performance.

In some embodiments, the processor is configured to calibrate the range of motion of the user by iteratively presenting training targets along an axis and recording positional limits for the movement.

In some embodiments, the processor is configured to store the training parameters in a remote database upon completion of the visual trial.

In another broad aspect, in accordance with some embodiments, there is generally provided a computer-implemented method for determining spatial positions of training targets for visual trials presented within an augmented reality environment; the method including generating a visual trial presented to a user via a display system, the display system having sensors configured to track movement of the user, the visual trial having at least one training target presented at a spatial position within the augmented reality environment; obtaining the movement of the user from the sensors of the display system during the visual trial; assessing a range of motion of the user by evaluating an alignment between the movement of the user and the spatial position of the training target during the visual trial to determine training parameters for a subsequent visual trial defining a subsequent spatial position for a subsequent training target; generating the subsequent visual trial presented to the user via the display system; and applying the training parameters to determine the subsequent spatial position of the subsequent training target within the augmented reality environment.

In some embodiments, the method further includes determining the spatial position for the subsequent training target by selecting a start position and an end position for the subsequent training target from within the range of motion limits.

In some embodiments, the method further includes generating a trajectory for the training target between the start position and the end position along a path generated from a combination of straight angular components and sinusoidally varying angular components.

In some embodiments, assessing the range of motion of the user further includes determining whether the movement is substantially aligned with the training target for a predetermined threshold time.

In some embodiments, the training parameters are determined based on tracked movement associated with the visual trial; the tracked movement defining one or more performance metrics.

In some embodiments, the performance metrics include a smoothness metric for the visual trial based on the movement; the smoothness metric corresponding to the range of motion assessed over a predetermined period of time.

In some embodiments, the performance metrics include a success indicator associated with the visual trial; the success indicator indicating whether the user successfully tracked the training target for at least a threshold hold time while the training target was moving.

In some embodiments, determining the training parameters for the subsequent visual trial includes evaluating the one or more performance metrics to determine a trial performance; and adjusting a training parameter of the subsequent training target in the subsequent visual trial based on the trial performance.

In some embodiments, the method further includes calibrating the range of motion of the user by iteratively presenting training targets along an axis and recording positional limits for the movement.

In some embodiments, the method further includes storing the training parameters in a remote database upon completion of the visual trial.

### BRIEF DESCRIPTION OF THE DRAWINGS

For a better understanding of the embodiments described herein and to show more clearly how they may be carried into effect, reference will now be made, by way of example only, to the accompanying drawings which show at least one exemplary embodiment, and in which:

FIG. 1A is an example diagram illustrating an example augmented reality environment, in accordance with some embodiments.

FIG. 1B is a block diagram illustrating an example training system, in accordance with some embodiments.

FIG. 2 is a device diagram illustrating an example server of the training system of FIG. 1B, in accordance with some embodiments.

FIG. 3 is a flow diagram illustrating an example method for determining spatial positions of training targets for visual trials presented within an augmented reality environment, in accordance with some embodiments.

FIG. 4 is a flow diagram illustrating an example method for implementing an example training system, in accordance with some embodiments.

FIG. 5 is a flow diagram illustrating an example method for implementing a training system with adaptive mode, in accordance with some embodiments.

FIG. 6A is a schematic diagram illustrating an example visual trial in which a user is guided to a training target presented within an augmented reality environment, in accordance with some embodiments.

FIG. 6B is another schematic diagram illustrating an example visual trial in which a training target moves along a path within an augmented reality environment and a user is guided to track the moving training target, in accordance with some embodiments.

FIG. 7A is a graph illustrating progression of range of motion over a plurality of training sessions.

FIG. 7B is a graph illustrating progression of movement smoothness over a plurality of training sessions.

FIG. 7C is a graph illustrating progression of performance score over a plurality of training sessions.

### DESCRIPTION OF VARIOUS EMBODIMENTS

Various embodiments in accordance with the teachings herein will be described below to provide an example of at least one embodiment of the claimed subject matter. No embodiment described herein limits any claimed subject matter. The claimed subject matter is not limited to devices, systems or methods having all of the features of any one of the devices, systems or methods described below or to features common to multiple or all of the devices, systems or methods described herein. It is possible that there may be a device, system or method described herein that is not an embodiment of any claimed subject matter. Any subject matter that is described herein that is not claimed in this document may be the subject matter of another protective instrument, for example, a continuing patent application, and the applicants, inventors or owners do not intend to abandon, disclaim or dedicate to the public any such subject matter by its disclosure in this document.

For simplicity and clarity of illustration, reference numerals may be repeated among the figures to indicate corresponding or analogous elements. In addition, numerous specific details are set forth in order to provide a thorough understanding of the subject matter described herein. However, it will be understood by those of ordinary skill in the art that the subject matter described herein may be practiced without these specific details. In other instances, well-known methods, procedures and components have not been described in detail so as not to obscure the subject matter described herein. The description is not to be considered as limiting the scope of the subject matter described herein.

It should also be noted that the terms “coupled” or “coupling” as used herein can have several different meanings depending in the context in which these terms are used. For example, the terms coupled or coupling can have a logical, mechanical, fluidic or electrical connotation. For example, as used herein, the terms coupled or coupling can indicate that two elements or devices can be directly connected to one another or connected to one another through one or more intermediate elements or devices via an electrical or magnetic signal, electrical connection, an electrical element or a mechanical element depending on the particular context. Furthermore, coupled electrical elements may send and/or receive data.

Unless the context requires otherwise, throughout the specification and claims which follow, the word “comprise” and variations thereof, such as, “comprises” and “comprising” are to be construed in an open, inclusive sense, that is, as “including, but not limited to”.

It should also be noted that, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

It should be noted that terms of degree such as “substantially”, “about” and “approximately” as used herein mean a reasonable amount of deviation of the modified term such that the end result is not significantly changed. These terms of degree may also be construed as including a deviation of the modified term, such as by 1%, 2%, 5% or 10%, for example, if this deviation does not negate the meaning of the term it modifies.

Furthermore, the recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed, such as 1%, 2%, 5%, or 10%, for example.

Reference throughout this specification to “one embodiment”, “an embodiment”, “at least one embodiment” or “some embodiments” means that one or more particular features, structures, or characteristics may be combined in any suitable manner in one or more embodiments, unless otherwise specified to be not combinable or to be alternative options.

As used in this specification and the appended claims, the singular forms “a,” “an,” and “the” include plural referents unless the content clearly dictates otherwise. It should also be noted that the term “or” is generally employed in its broadest sense, that is, as meaning “and/or” unless the content clearly dictates otherwise.

Similarly, throughout this specification and the appended claims the term “communicative” as in “communicative pathway,” “communicative coupling,” and in variants such as “communicatively coupled,” is generally used to refer to any engineered arrangement for transferring and/or exchanging information. Exemplary communicative pathways include, but are not limited to, electrically conductive pathways (e.g., electrically conductive wires, electrically conductive traces), magnetic pathways (e.g., magnetic media), optical pathways (e.g., optical fiber), electromagnetically radiative pathways (e.g., radio waves), or any combination thereof. Exemplary communicative couplings include, but are not limited to, logical couplings, electrical couplings, magnetic couplings, optical couplings, radio couplings, or any combination thereof.

Throughout this specification and the appended claims, infinitive verb forms are often used. Examples include, without limitation: “to detect,” “to provide,” “to transmit,” “to communicate,” “to process,” “to route,” and the like. Unless the specific context requires otherwise, such infinitive verb forms are used in an open, inclusive sense, that is as “to, at least, detect,” to, at least, provide,” “to, at least, transmit,” and so on.

The example systems and methods described herein may be implemented as a combination of hardware or software. In some cases, the examples described herein may be implemented, at least in part, by using one or more computer programs, executing on one or more programmable devices comprising at least one processing element, and a data storage element (including volatile memory, non-volatile memory, storage elements, or any combination thereof). These devices may also have at least one input device (e.g. a keyboard, mouse, touchscreen, or the like), and at least one output device (e.g. a display screen, a printer, a wireless radio, or the like) depending on the nature of the device.

Some elements that are used to implement at least part of the systems, methods, and devices described herein may be implemented via software that is written in a high-level procedural language such as object-oriented programming. The program code may be written in C⁺⁺, C #, JavaScript, Python, or any other suitable programming language and may comprise modules or classes, as is known to those skilled in object-oriented programming. Alternatively, or in addition thereto, some of these elements implemented via software may be written in assembly language, machine language, or firmware as needed. In either case, the language may be a compiled or interpreted language.

At least some of these software programs may be stored on a computer readable medium such as, but not limited to, a ROM, a magnetic disk, an optical disc, a USB key, and the like that is readable by a device having at least one processor, an operating system, and the associated hardware and software that is used to implement the functionality of at least one of the methods described herein. The software program code, when read by the device, configures the device to operate in a new, specific, and predefined manner (e.g., as a specific-purpose computer) in order to perform at least one of the methods described herein.

Furthermore, at least some of the programs associated with the systems and methods described herein may be capable of being distributed in a computer program product including a computer readable medium that bears computer usable instructions for one or more processors. The medium may be provided in various forms, including non-transitory forms such as, but not limited to, one or more diskettes, compact disks, tapes, chips, and magnetic and electronic storage. Alternatively, the medium may be transitory in nature such as, but not limited to, wire-line transmissions, satellite transmissions, internet transmissions (e.g. downloads), media, digital and analog signals, and the like. The computer useable instructions may also be in various formats, including compiled and non-compiled code.

Prevailing approaches for rehabilitation, exercise guidance, and therapeutic movement training often rely on immersive virtual reality systems, externally instrumented motion-analysis arrangements, or therapist-led programming of prescribed movement activities. Although such approaches may provide visual instruction or guided activity, they can be cumbersome to deploy, may depend on specialized equipment or clinical oversight, and may be less practical for frequent use in everyday settings.

It would therefore be preferable to provide a training and therapy-related platform that can be used in a more accessible and self-directed manner, while still supporting guided activity within an augmented environment. It would further be preferable for such a platform to be suitable for deployment outside of a clinic, including in a home setting, so that a user can engage in the activity without requiring a physician or other clinician to be physically present for each session.

Systems implemented using augmented reality may offer practical advantages over approaches that rely on fully immersive virtual reality equipment to accomplish similar goals. For example, augmented reality platforms can be implemented using comparatively lightweight, ergonomic, and portable hardware, which may be easier for a user to wear and incorporate into routine activity. Because digital content is presented in connection with the user's real-world surroundings rather than replacing the surroundings entirely, the experience may also feel more natural and comfortable for extended use, thereby supporting continued participation.

The various embodiments disclosed herein address these issues by providing augmented reality-based systems and methods that present guided visual activity within a user's environment in a way that is portable, accessible, and suitable for repeated use. In various embodiments, the system may support individualized use without requiring externally mounted sensing arrangements, therapist-authored movement demonstrations, or continual in-person supervision, thereby reducing deployment complexity while making the system easier to use in non-clinical environments.

In various embodiments, the system may be arranged to support user-specific guided activity within comfortable movement limits and to provide visual interaction in a manner that can reflect the needs or abilities of a particular user over time. This can assist in making the experience more appropriate for home use, where convenience, simplicity, and repeatability may be important to sustained participation. It can also reduce the amount of clinician time that might otherwise be needed to initially tailor therapeutic activity for a user or to repeatedly revise that activity over successive sessions.

In this way, the disclosed embodiments can provide a practical alternative to heavier, more clinic-dependent rehabilitation or therapy platforms by combining portability, user comfort, guided activity, and an engaging presentation format. The result can be a system that is more suitable for regular use in everyday environments, including home environments, while still supporting therapeutic, rehabilitative, exercise, or other training-related applications.

The various embodiments disclosed herein address these issues by providing training systems and methods in which a display system presents visual trials within an augmented reality environment and one or more sensors track movement of a user during the visual trials. Based on an evaluation of alignment between the user's movement and a presented training target, the system can assess a range of motion of the user and determine one or more training parameters for a subsequent visual trial, including a subsequent spatial position for a subsequent training target.

In various embodiments, the system can calibrate the user's range of motion by iteratively presenting training targets along one or more axes and recording positional limits for the user's movement. The system can then determine start and end positions for subsequent training targets from within range-of-motion limits of the user and generate a target trajectory between the start position and the end position. In some embodiments, the trajectory can be generated from a combination of straight angular components and sinusoidally varying angular components, thereby enabling controlled variation in target motion while maintaining the target within a bounded augmented reality training space.

In further embodiments, the system can determine whether the user's movement is aligned with a training target for at least a predetermined threshold time, derive one or more performance metrics such as a smoothness metric for a visual trial, determine a baseline smoothness metric over a plurality of visual trials, and adjust a movement speed of a subsequent training target when the smoothness metric deviates from the baseline smoothness metric by at least a predetermined threshold. In some embodiments, one or more training parameters may also be stored in a remote database upon completion of a visual trial or training session, thereby supporting longer-term tracking and review of user performance.

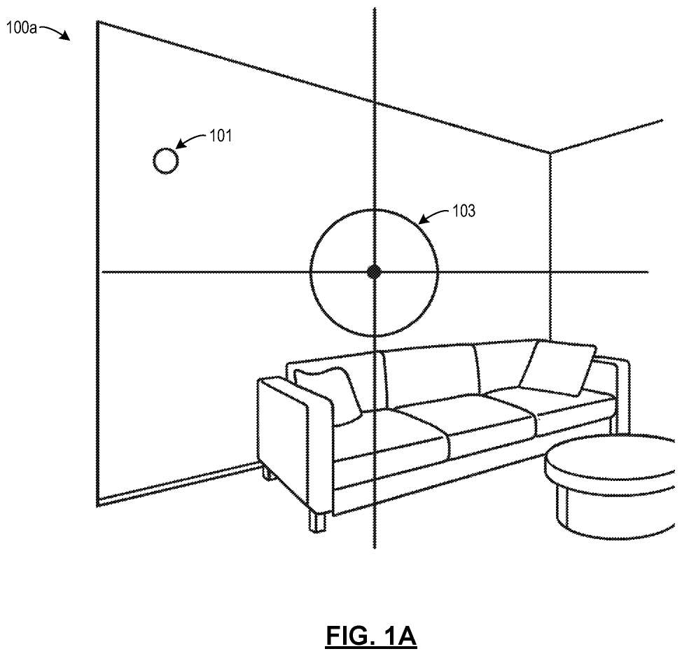

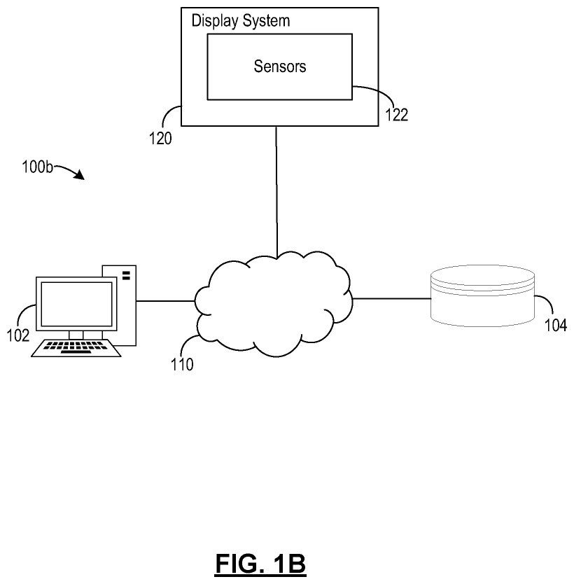

Reference is next made to FIG. 1A, which illustrates an example augmented reality environment 100a in accordance with some embodiments. In this example, the system may present visual content within the user's real-world surroundings so that the user can engage in guided movement while remaining aware of the physical environment around them. A user may interact with the system by performing specific movements in response to targets being displayed. In some embodiments, the target may appear as a simple visual object, such as an orb 101, and a visual guide 103 may be shown within the user's view to help the user understand where to direct their gaze or movement. In this manner, the augmented reality environment 100a may provide a guided and interactive experience that encourages the user to perform repeated movements in a structured way.

In some embodiments, the system may be implemented using an augmented reality headset or glasses worn by the user. The augmented reality headset may present visual targets within the user's field of view while the user remains in a familiar physical setting. In this example, the system may be used to direct the user to perform guided head and neck movements as part of an activity intended to support improvement in neck mobility. For example, the user may wear the augmented reality headset or glasses and follow displayed targets by turning or repositioning their head and neck in different directions. A target may first appear to one side of the user's view, prompting the user to move their neck to direct their vision toward that location. Upon successfully directing their vision to the first target, another target may appear at another location within the user's view, prompting them to redirect their vision. As the user continues interacting with the system over repeated trials or sessions, the presented targets may encourage the user to perform a variety of neck movements in different directions. This repeated guided interaction may help the user practice mobility in a progressive manner and may support improvement over time.

Reference is next made to FIG. 1B, which provides a general overview of a training system 100b. In accordance with some embodiments of the disclosed invention, the training system 100b may be configured to present visual trials within an augmented reality environment and to track movement of a user during the visual trials. The training system 100b includes a server 102, a database 104, a network 110, and a display system 120 having one or more sensors. The server 102, the database 104, and the display system 120 may be connected to each other through the network 110.

The server 102 may be configured to control operation of the training system 100b. In some embodiments, the server 102 may comprise one or more processors and a memory storing instructions for carrying out one or more operations of the training system 100b. For example, the server 102 may be configured to cause the display system 120 to present a visual trial to a user within an augmented reality environment, receive sensor data indicative of movement of the user, assess the movement of the user during the visual trial, and determine one or more training parameters for a subsequent visual trial. In some embodiments, the server 102 may further be configured to determine a spatial position for a subsequent training target, apply one or more training parameters to a subsequent visual trial, and coordinate presentation of the subsequent visual trial by the display system 120.

In some embodiments, the server 102 may be configured to support calibration and training operations for the user. For example, the server 102 may iteratively control presentation of visual content for establishing user-specific movement limits and may thereafter use information derived from prior user interaction to guide later-presented or subsequent visual trials. In some embodiments, the server 102 may also determine or update settings associated with movement range, timing, target behavior, or other training-related conditions for use during a session or across multiple sessions.

The database 104 may be configured to store, organize, and retrieve information associated with operation of the training system 100b. In some embodiments, the database 104 may store training parameters, session settings, user-specific movement information, performance metrics, trial results, and historical session records. In some embodiments, the database 104 may further store summary information upon completion of a visual trial or training session so as to support later review, longitudinal tracking, or remote access to stored records. The database 104 may be implemented as a local database, a remote database, a cloud-based database, or any other suitable digital storage arrangement.

The network 110 may be any network capable of carrying data between the elements shown in FIG. 1B. In some embodiments, the network 110 may communicatively couple the server 102, the database 104, and the display system 120 such that information associated with presentation of visual trials, sensed user movement, and stored training information can be exchanged between those elements. The network 110 may include one or more wired or wireless communication links and may be implemented using any suitable communication infrastructure for enabling operation of the training system 100b.

The display system 120 may be any display system capable of displaying an augmented reality environment to the user. In some embodiments, the display system 120 may comprise augmented reality glasses, a head-mounted display, or another display platform configured to present digital visual content in connection with the user's surrounding environment. The display system 120 includes one or more sensors configured to measure movement of the user. In some embodiments, the one or more sensors may provide sensor data to the server 102 for use in carrying out one or more training operations, including assessment of user movement during a visual trial and presentation of subsequent visual content.

In operation, the training system 100b may present one or more visual trials to the user through the display system 120 while obtaining information about movement of the user from the one or more sensors of the display system 120. The server 102 may use this information to support operation of the training system 100b during a current visual trial and, in some embodiments, to support preparation of a subsequent visual trial. Information generated during operation of the training system 100b may be stored in the database 104 and communicated between system elements through the network 110.

The one or more sensors 122 may be integrated with, mounted to, or otherwise form part of the display system 120 and may be configured to measure movement of the user during operation of the training system 100b. In some embodiments, the one or more sensors 122 may comprise one or more motion-related sensors, position-related sensors, orientation-related sensors, imaging sensors, inertial sensors, or combinations thereof. For example, the one or more sensors 122 may include one or more cameras, accelerometers, gyroscopes, magnetometers, depth sensors, or other sensors capable of generating sensor data indicative of movement, position, orientation, or spatial relationship of the display system 120 relative to the user or the surrounding environment. The one or more sensors 122 may provide such sensor data to the server 102 for use in carrying out one or more operations of the training system 100b.

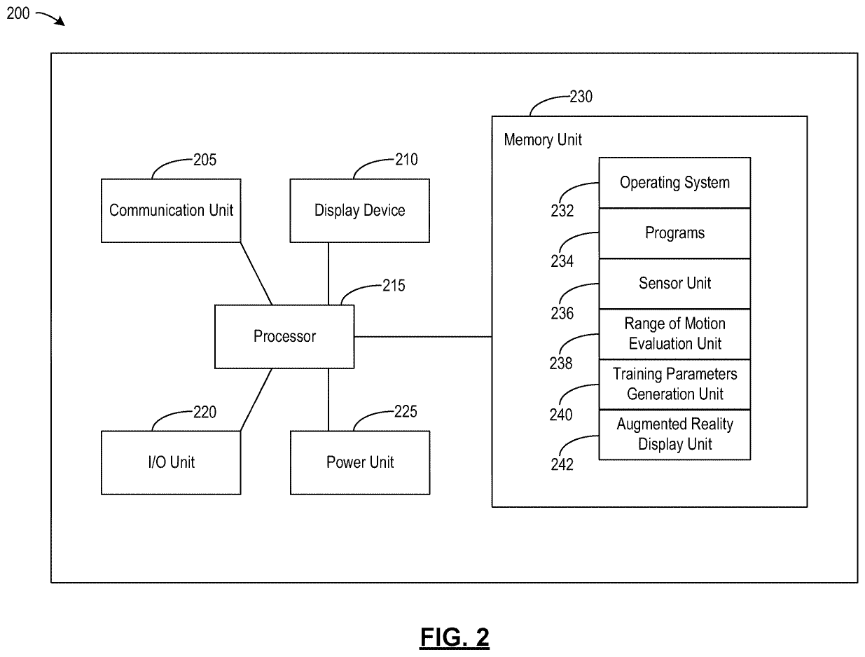

Reference is next made to FIG. 2, which shows a device diagram 200 of an example server 102 in accordance with one or more embodiments. The server 102 can include a communication unit 205, a display device 210, a processor 215, an I/O unit 220, a power unit 225, and a memory unit 230.

The communication unit 205 can include any combination of hardware that enables wired or wireless communication by the server 102. For example, the communication unit 205 may include a radio, network card, wireless transceiver, port interface, or other communication hardware suitable for exchanging data with the display system 120 and its sensors 122, the database 104, and other computing devices through the network 110. In some embodiments, the display system 120 can include augmented reality glasses configured to communicate with a smartphone through a USB-C connection. The smartphone can execute a downloaded software application associated with the training system 100b, and the software application can communicate with the server 102 over a wireless network, such as a Wi-Fi network, to exchange sensor-related information, transmit instructions, and access stored training-related data. The communication unit 205 can allow the server 102 to receive sensor-related information, transmit instructions, and exchange stored training-related data with other elements of the training system 100b.

The display device 210 may include any suitable visual interface configured to present operational information, training-related information, system states, configuration options, and user-interactive elements. In various embodiments, the display device 210 can comprise a monitor, touchscreen, integrated panel, laptop display, tablet screen, or any other electronic visual output capable of rendering graphical or text-based information associated with operation of the training system 100b. In some embodiments, the display device 210 is distinct from the display system 120 used to present the actual training content within the augmented reality environment. For example, the display system 120 can include augmented reality hardware configured to present visual trials and training targets to the user, while the display device 210 can be associated with a smartphone or other computing device configured to present session information, setup menus, calibration options, training settings, trial summaries, movement-related information, or stored records associated with prior sessions. Accordingly, the display device 210 may support configuration, monitoring, and review of training operations, while the display system 120 presents the training itself.

In some embodiments, the display device 210 may be integrated with, or operate in conjunction with, augmented-reality technology. For example, the display device 210 may be implemented as, or communicate with, augmented reality glasses, a head-mounted display, or another display platform configured to present digital content within the user's real-world environment. In such embodiments, the display device 210 may support presentation of training content in an augmented reality environment while also providing a user with a visual experience that remains connected to the surrounding physical environment.

The processor 215 controls operation of the server 102. The processor 215 can be any suitable processor, controller, or digital processing device that provides sufficient processing power for operation of the training system 100b. In some embodiments, the processor 215 may execute instructions for causing presentation of visual trials, receiving and evaluating movement-related information, determining one or more training parameters, and supporting display of visual content within an augmented reality environment. The processor 215 may include one or more processors, and in some embodiments different processors may be configured to perform different dedicated tasks.

The I/O unit 220 can include hardware for interfacing with at least one of a mouse, a keyboard, a touchscreen, a thumbwheel, a trackpad, a trackball, a card-reader, an audio source, a microphone, voice-recognition software, and the like, depending on the particular implementation of the server 102. The I/O unit 220 may facilitate user input to the server 102, including for example entry of user information, session settings, calibration selections, training selections, or review commands associated with stored records. In some cases, one or more of these components can be integrated with one another.

The power unit 225 can be any suitable power source that provides power to the server 102, such as a power adaptor, an internal power supply, or a rechargeable battery pack, depending on the implementation of the server 102. In some embodiments, the power unit 225 may support portable or home-based deployment of the training system 100b by providing power in a form suitable for non-clinical use environments.

The memory unit 230 comprises software code for implementing, among other programs 232, an operating system 234, a sensor unit 236, a range of motion evaluation unit 238, a training parameters generation unit 240, and an augmented reality display unit 242. The memory unit 230 may include RAM, ROM, one or more hard drives, one or more flash drives, or other suitable data storage elements. The operating system 234 may manage execution of software instructions and coordination of hardware resources, while the one or more programs 232 may include application logic associated with calibration, training, presentation of visual trials, storage of session information, and other operations of the training system 100b.

The sensor unit 236 may comprise executable instructions, interface logic, and data structures that, when executed by the processor 215, cause the processor 215 to receive, organize, and interpret input from one or more sensors associated with the display system. In some embodiments, the sensor unit 236 may receive sensor data indicative of movement, position, orientation, or spatial relationship of the display system relative to the user or the surrounding environment. For example, in some embodiments, where the display system includes augmented reality glasses having one or more sensors, the sensor unit 236 may receive movement data associated with movement of the user's head and neck while the user engages with visual content presented in the augmented reality environment. In other examples, the sensor may be a camera configured to track the movement of the user's limbs. In some embodiments, such movement data may be used during calibration operations, training operations, or both.

The range of motion evaluation unit 238 may comprise executable instructions and evaluation logic that, when executed by the processor 215, cause the processor 215 to assess a range of motion of the user based on movement-related information obtained during one or more visual trials. In some embodiments, the range of motion evaluation unit 238 may evaluate an alignment between movement of the user and a presented training target to assess the range of motion of the user. For example, during a calibration sequence, the range of motion evaluation unit 238 may assess user-specific movement limits by iteratively presenting visual content along one or more directions and recording positional limits associated with leftward, rightward, upward, or downward movement. In some embodiments, the range of motion evaluation unit 238 may also determine whether the user remains aligned with presented visual content for at least a predetermined threshold time and may use that information as part of the range of motion assessment.

The training parameters generation unit 240 may comprise executable instructions and generation logic that, when executed by the processor 215, cause the processor 215 to determine one or more training parameters for a subsequent visual trial. In some embodiments, the one or more training parameters may define a subsequent spatial position for a subsequent training target, a movement speed, a hold time, a movement pattern, or one or more range-related settings for use in a later-presented or subsequent visual trial. For example, the training parameters generation unit 240 may determine a start position and an end position from within user-specific movement limits and may generate a trajectory between those positions. In some embodiments, the trajectory may include a combination of straight angular components and sinusoidally varying angular components. In further embodiments, the training parameters generation unit 240 may determine one or more performance metrics, including a smoothness metric, compare the smoothness metric to a baseline smoothness metric determined over a plurality of visual trials, and adjust a movement speed for a subsequent visual trial when there is a difference between the smoothness metric and the baseline smoothness metric by at least a predetermined threshold.

In some embodiments, the training parameters generation unit 240 may additionally adjust a direction of movement for the subsequent visual trial based on the smoothness metric and/or other performance metrics associated with one or more prior visual trials. For example, when the processor 215 determines that movement in a particular direction is comparatively more challenging for the user, such as head rotation to the right, the training parameters generation unit 240 may cause subsequent training targets to be positioned or moved more frequently in that direction so as to provide additional training emphasis for that side or movement pattern.

The augmented reality display unit 242 may comprise executable instructions and display-generation logic that, when executed by the processor 215, cause the processor 215 to apply one or more training parameters to an augmented reality environment for presentation to the user. In some embodiments, the augmented reality display unit 242 may control where visual content appears within the user's field of view, how such visual content moves within the augmented reality environment, the direction in which such visual content moves, and when subsequent visual content is presented. For example, the augmented reality display unit 242 may cause a training target to appear at a selected start position, move toward an end position at a selected speed, and follow a linear, sinusoidal, or other movement pattern defined for a given visual trial. In some embodiments, the augmented reality display unit 242 may further support visual feedback to the user during interaction with the presented content and may coordinate presentation of subsequent visual trials based on training-related settings generated by the training parameters generation unit 240.

During operation, the processor 215 may execute the instructions stored in the memory unit 230 so that the sensor unit 236, the range of motion evaluation unit 238, the training parameters generation unit 240, and the augmented reality display unit 242 cooperate to support operation of the training system 100b. For example, sensor-related information obtained from the display system may be used to assess user movement, one or more training parameters may then be determined for a subsequent visual trial, and visual content may be presented in the augmented reality environment according to those training parameters. In some embodiments, information associated with a visual trial or training session may also be stored for later access or review.

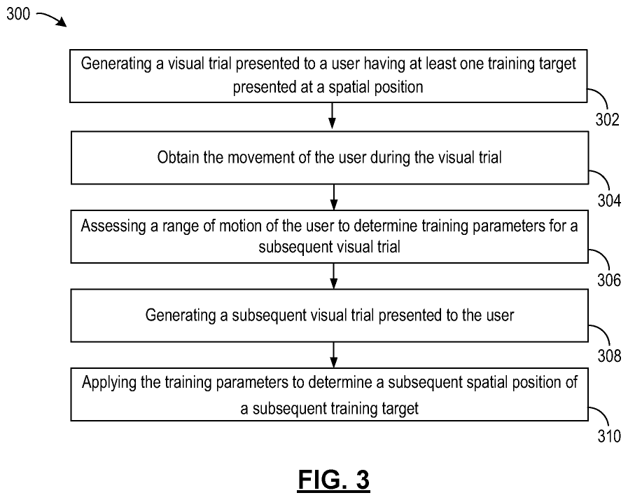

Reference is next made to FIG. 3, which shows a flow diagram of an example method 300 in accordance with some embodiments. Method 300 describes a process for determining spatial positions of training targets for visual trials presented within an augmented reality environment. In some embodiments, the method 300 is carried out using a display system having one or more sensors configured to track movement of a user. For example, in some embodiments, the method can be implemented as an augmented reality headset configured to present training targets in the user's field of view.

The method 300 begins at 302 with generating a visual trial presented to a user via a display system. The display system may have one or more sensors configured to track movement of the user. The visual trial may have at least one training target presented at a spatial position within the augmented reality environment. In some embodiments, the display system may comprise an augmented reality headset for presenting the visual trial.

In some embodiments, step 302 may also include a calibration operation. The calibration operation may iteratively present training targets along an axis and record positional limits for the movement. For example, the headset may present successive training targets in leftward, rightward, upward, and downward directions. The user may align with each training target in sequence while the system records positional limits associated with those movements.

The method proceeds to 304 with obtaining the movement of the user during the visual trial. In some embodiments, obtaining the movement of the user may comprise obtaining tracked movement data from one or more sensors of the display system during the visual trial. The tracked movement data may be indicative of movement of the user relative to the augmented reality environment. For example, where the display system comprises an augmented reality display system, the one or more sensors may generate data indicative of position, orientation, or motion of the headset as the user moves to align with a displayed training target. In some embodiments, the obtained movement data may correspond to head and neck movement of the user during the visual trial.

In some embodiments, the tracked movement data obtained at 304 may be used to determine how the user is moving relative to the spatial position of the training target presented during the visual trial. For example, the display system may present an orb within the augmented reality environment, and the system may obtain movement data indicating how the user changes pitch, yaw, or roll while moving to align with the orb. In some embodiments, the tracked movement data may be obtained continuously or periodically during the visual trial so that later processing can evaluate whether the movement of the user is aligned with the training target and can further assess the range of motion of the user for determining training parameters for a subsequent visual trial.

The method proceeds to 306 with assessing a range of motion of the user. The range of motion may be assessed by evaluating an alignment between the movement of the user and the spatial position of the training target during the visual trial. The assessment at step 306 may be used to determine training parameters for a subsequent visual trial. In some embodiments, the one or more sensors of the display system may generate movement data indicative of position, orientation, or motion of the user during the visual trial. The method may compare that movement data to the displayed position of the training target. The system may then determine whether the user is remaining aligned with the training target during the visual trial.

In some embodiments, assessing the range of motion may further comprise determining whether the movement is substantially aligned with the training target for a predetermined threshold time. For example, the method may determine whether the center of the headset view is maintained over the training target for at least a selected hold time. For example, a training target may be presented in an augmented reality environment, and the system may determine whether movement of the user is substantially aligned with the training target for at least that threshold time. In some embodiments, movement of the user may be considered substantially aligned with the training target when a crosshair, fixed relative to the user's field of view, is positioned within bounds defined by the training target. For example, where the training target is rendered as an orb, the system may determine that substantial alignment is present when the crosshair lies within the visible perimeter of the orb and remains within that perimeter for at least the predetermined threshold time.

The display system may include one or more sensors that generate movement data indicative of the position and orientation of the display system as the user moves. A processor may use that movement data to determine whether a visual indicator, such as a crosshair fixed relative to the user's field of view, is aligned with the displayed training target. When the crosshair is brought into alignment with the training target, the system may start a timer. If alignment is maintained continuously until the timer reaches the predetermined threshold time, the system may treat the trial as successfully completed. If alignment is lost before the threshold time expires, the timer may be reset or paused, and the user may be required to realign with the training target.

In some embodiments, the crosshair may be positioned centrally in the user's field of view and may remain fixed relative to the display system so that it moves with the user. This arrangement may provide the user with a visual guide that indicates where the center of the user's view is directed within the augmented reality environment. The user may therefore move to bring the crosshair onto the training target, rather than trying to estimate alignment without visual assistance. In addition, the crosshair may provide the user with feedback regarding head position and orientation in space. For example, the crosshair may assist the user in recognizing whether the head is tilted to the left or right, or is otherwise improperly positioned relative to an intended posture. In some embodiments, the system may provide additional feedback when alignment occurs. For example, when the crosshair is aligned with the training target, the training target may change color, such as from red to green, to indicate that the user is correctly positioned and that the hold timer is running. This can make it easier for the user to understand when alignment has been achieved and when the user should continue holding position until the threshold time has elapsed. The crosshair may therefore also assist the user in correcting improper head and neck posture during the training session.

Referring back to FIG. 3, the method proceeds to 308 with generating a subsequent visual trial presented to the user via the display system. The subsequent visual trial may have a subsequent training target. In some embodiments, the subsequent visual trial may be configured using the training parameters determined at step 306. The subsequent training target may therefore be presented at a position, within a movement region, or with a motion behavior selected for that user based on the range of motion demonstrated during a previous trial. For example, after a first visual trial is presented and the user's movement is assessed, a subsequent orb may be presented within user-specific movement limits established during the visual trial or during a calibration phase conducted prior to the visual trial. In some embodiments, the subsequent visual trial may be configured using tracked movement information and one or more performance metrics from one or more prior visual trials. The subsequent trial may therefore reflect previously observed movement capability while remaining within a user-appropriate movement space.

The method proceeds to 310 with applying the training parameters to the display system presenting the subsequent visual trial. The training parameters may be applied to determine the subsequent spatial position of the subsequent training target within the augmented reality environment. In some embodiments, determining the subsequent spatial position may comprise selecting a start position and an end position for the subsequent training target from within the range of motion limits of the user. For example, a training target may be assigned a start position in an upper-left region of the user's comfortable movement space and an end position in a lower-right region of that movement space. In some embodiments, a trajectory for the training target may be generated between the start position and the end position. The trajectory may be along a path generated from a combination of straight angular components and sinusoidally varying angular components. For example, the training target may move generally across the field of view while also following a wave-like path.

In some embodiments, determining the training parameters for the subsequent visual trial may further comprise comparing the smoothness metric for the visual trial to the baseline smoothness metric to determine a difference between the smoothness metric and the baseline smoothness metric. For example, the baseline smoothness metric may be determined based on an average smoothness metric from a plurality of prior visual trials, such as the preceding twenty visual trials. In further embodiments, the baseline smoothness metric may be determined independently for each of a plurality of movement directions, such as rightward movement, leftward movement, downward movement (e.g., cervical flexion), and upward extension (e.g., cervical extension), such that performance for each movement direction is evaluated relative to a direction-specific smoothness baseline. The movement speed of the subsequent training target may then be adjusted based on that difference. For example, where the user's recent movement is smoother than the baseline, the subsequent training target may move at a greater speed. Where the user's recent movement is less smooth than the baseline, the subsequent training target may move at a lower speed. In some embodiments, the selected training parameters may also include hold-related settings or other movement-related settings for the subsequent visual trial.

In some embodiments, the one or more performance metrics may comprise a success indicator associated with the visual trial. The success indicator may indicate whether the user successfully tracked the training target for at least a threshold hold time while the training target was moving. For example, the threshold hold time may correspond to a minimum duration for which the user is to maintain alignment with, fixation on, or tracking of the moving training target in order for the visual trial to be identified as successful. In some embodiments, the threshold hold time may be predetermined, may be selected by a clinician, or may be adjusted based on one or more prior visual trials. Where the user maintains tracking of the moving training target for at least the threshold hold time, the success indicator may identify the visual trial as successful. Where the user does not maintain tracking of the moving training target for at least the threshold hold time, the success indicator may identify the visual trial as unsuccessful.

In some embodiments, to determine the training parameters for a subsequent visual trial, the processor may be configured to evaluate one or more performance metrics associated with a current visual trial to determine a trial performance. The one or more performance metrics may include, for example, a smoothness metric, a success indicator, or any combination thereof. The trial performance may correspond to an assessment of how well the user performed during the visual trial and may be used by the system to adjust a training parameter of a subsequent training target in a subsequent visual trial. For example, based on the trial performance, the system may increase, decrease, or maintain a difficulty of the subsequent visual trial by adjusting one or more training parameters such as movement speed, movement direction, hold time, target displacement, target trajectory, or another parameter associated with presentation of the subsequent training target.

The trial performance may be associated with a scoring system. For example, the processor may assign a score to the visual trial based on whether the user satisfied one or more of the performance metrics for that visual trial. In some examples, where the user achieves a smoothness metric satisfying a threshold criterion and/or the success indicator indicates that the user successfully tracked the training target for at least a threshold hold time while the training target was moving, the processor may award one or more points for the visual trial. The awarded score may define, or may form part of, the trial performance. Conversely, where the user does not satisfy the threshold criterion for the smoothness metric and/or does not successfully track the training target for at least the threshold hold time, the processor may assign a reduced score, no score, or another value indicative of lower performance.

The scoring system may be used to adapt the subsequent visual trial. For example, where the score associated with the trial performance indicates that the user successfully satisfied the one or more performance metrics, the processor may adjust the subsequent visual trial to increase difficulty, such as by increasing movement speed, increasing the threshold hold time, increasing target displacement, or selecting a more challenging movement direction. Where the score indicates that the user did not satisfy the one or more performance metrics, the processor may decrease difficulty, maintain difficulty, or modify the subsequent visual trial to emphasize a movement direction associated with lower performance. In this way, the scoring system may provide a quantitative representation of trial performance that can be used by the processor to adapt training to the user's demonstrated abilities.

In some embodiments, the method may further comprise storing the training parameters in a remote database upon completion of the visual trial. The stored information may include user-specific movement limits, selected training target positions, movement speeds, hold-related settings, performance-related information, or session-related information. For example, such information may be uploaded after completion of a visual trial or training session so that it can be retrieved later for review or use in a subsequent session. In this manner, method 300 may support repeated augmented reality training sessions in which training target-based visual trials are presented through an augmented reality headset and later trials are informed by information obtained from earlier user interaction.

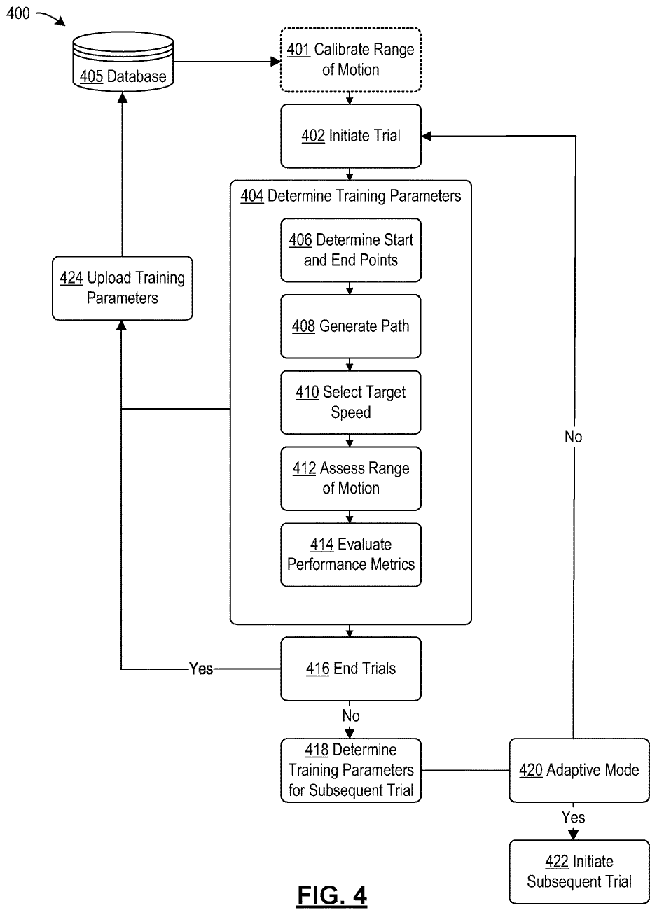

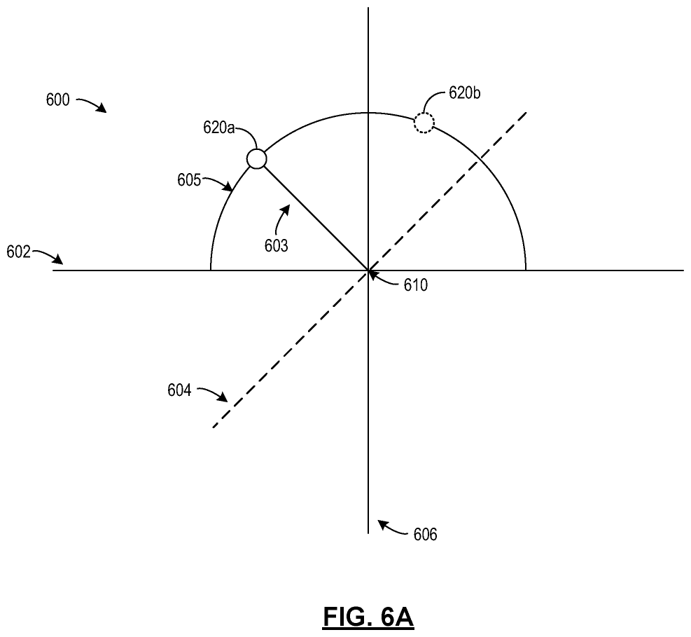

Reference is next made to FIG. 4, which shows a flow diagram of an example method 400 in accordance with some embodiments. Method 400 may be used to optionally calibrate a range of motion of a user, initiate a visual trial, and determine training parameters for later visual trials presented within an augmented reality environment. In some embodiments, the visual trial initiated by method 400 may correspond to the visual trial 600 shown in FIG. 6A.

In the examples described herein, the user may be described as moving his or her head and neck to align with or track a displayed training target. In such embodiments, the display system comprises an augmented reality headset and the training targets are presented within the user's field of view. However, it will be understood that the disclosed systems and methods are not limited to head and neck movement. In some embodiments, the disclosed systems and methods may be applied to other types of bodily movement, including movement of an arm, a hand, a shoulder, a torso, a leg, a foot, or combinations thereof.

In such embodiments, the display system and associated sensing arrangement may be configured to measure movement of the relevant body portion and to present training targets in a manner that guides the user through goal-directed movement of that body portion. For example, training targets may be positioned and moved so as to guide reaching movements of an arm, stepping or balance-related movements of a leg, upper-body rotational movement of the torso, or other therapeutic, rehabilitative, exercise, sports-training, or movement-training activity. The same general principles described herein may therefore be used to assess movement capability, determine training parameters for later visual trials, and adapt presentation of subsequent training targets for body regions other than the head and neck.

The method 400 may optionally begin at 401 with calibrating a range of motion of a user. In some embodiments, calibration may be performed by iteratively presenting training targets along an axis and recording positional limits for movement of the user. For example, an augmented reality headset may present orb-like training targets successively in leftward, rightward, upward, and downward directions while the user moves to align with each orb. The recorded positional limits may define user-specific movement limits for later visual trials.

In some embodiments, the database 405 may be used in connection with the calibration at 401. The database 405 may store calibration-related information for the user, including recorded positional limits, user-specific range-of-motion limits, selected calibration settings, or other parameters derived during the calibration process. For example, where the calibration is performed by iteratively presenting orb-like training targets in leftward, rightward, upward, and downward directions, the resulting positional limits for those directions may be stored in the database 405. In some embodiments, the stored calibration information may be retrieved when initiating a later trial at 402 so that one or more training parameters for the trial can be based on previously determined movement limits of the user. The database 405 may therefore allow calibration settings to persist across sessions and may reduce the need to repeat the full calibration process before each subsequent training session.

In some embodiments, the calibration at 401 may define the environment shown in FIG. 6A, which illustrates an example visual trial 600. The augmented reality environment may be defined relative to a coordinate system having a first axis 602, a second axis 604, and a third axis 606. In some embodiments, the first axis 602 may correspond to an x-axis, the second axis 604 may correspond to a y-axis, and the third axis 606 may correspond to a z-axis. The user 610 may generally remain in substantially the same position within the environment while moving to align his or her view with one or more training targets presented within the environment. In this manner, the position of the user 610 can serve as a reference point from which spatial positions of training targets may be defined in three dimensions.

The user 610 may generally remain centered within that environment while moving to align his or her view with displayed training targets. In some embodiments, the recorded movement limits from calibration may define what portion of that environment is available for presentation of the training targets. In some embodiments, movement of the user 610 within the visual trial 600 may be described in relation to pitch, yaw, and roll. For example, where a training target appears to the left or right of the user 610, the user 610 may adjust yaw to align with the training target. Where a training target appears above or below the user 610, the user 610 may adjust pitch to align with the training target. In some embodiments, roll may also vary as the user 610 naturally tilts or rotates relative to the displayed environment while aligning with a training target. Although the user 610 may remain substantially centered in position, the orientation of the user's head, neck, or view may change in three dimensions so that the displayed training target can be brought into alignment with the user's field of view.

At 402, the method may initiate a visual trial. In some embodiments, initiating the visual trial may include presenting a training target 620a within the visual trial 600 of FIG. 6A. The training target 620a may be rendered as an orb. The orb may be displayed at a spatial position defined relative to the axes 602, 604, and 606. The user 610 may move to align with the orb while generally remaining in the same physical position. For example, the user may adjust yaw when the orb appears left or right. The user may adjust pitch (e.g., through cervical flexion and extension) when the orb appears above or below. The user may also exhibit roll as part of natural head movement during alignment. The training target 620a may initially appear at a location on the path 605 that requires the user 610 to rotate leftward, rightward, upward, or downward to align with the training target 620a. In some embodiments, the user 610 may be guided to the training target 620a by a visual indicator, such as a crosshair fixed relative to the user's field of view, so that the user can move until the crosshair is aligned with the orb.

A radius 603 may extend between the user 610 and a displayed training target. In some embodiments, the radius 603 may define a distance between the user 610 and the training target that is fixed for a given visual trial. For example, the training target may be displayed at a substantially constant distance from the user 610 while the spatial position of the training target changes along one or more angular directions. In other embodiments, the radius 603 may be altered. For example, the radius 603 may be increased to place the training target farther from the user 610 or decreased to place the training target closer to the user 610.

The visual trial 600 may further include a path 605. The path 605 may correspond to a path or line on which a training target can appear while remaining a fixed distance from the user 610. In some embodiments, the path 605 may be defined at the radius 603 from the user 610 so that points along the path 605 are positioned on a surface that is a selected distance from the user 610. For example, the path 605 may correspond to a curved path, arc, ring segment, spherical path, or other locus of points at a common distance from the user 610. In this manner, the training target may be moved or positioned along the path 605 without necessarily changing its distance from the user 610.

The visual trial 600 may be used to assess a range of motion of the user 610. For example, movement of the user 610 relative to the training target 620a may be measured to determine whether the user 610 can align with the training target 620a and, in some embodiments, whether that alignment can be maintained for a predetermined threshold time. The assessed movement may be used to identify user-specific movement limits in one or more directions. For example, the assessed movement may indicate comfortable or achievable yaw limits, pitch limits, or combinations thereof for the user 610. In some embodiments, the assessed range of motion may be used to determine one or more training parameters for a subsequent visual trial.

At 404, the method may determine one or more training parameters for the trial. In some embodiments, the training parameters may be based at least in part on the calibration performed at 401. For example, the training parameters may include one or more range-related settings derived from the user-specific movement limits recorded during calibration. The training parameters may also include a selected radius 603, a selected path 605, a start position, an end position, a movement speed, a hold time, a movement pattern, or a target size.

In some embodiments, the path 605 defined by the radius 603 may correspond to a training parameter that can be adjusted for a subsequent visual trial. The radius 603 may define the distance between the user 610 and a training target displayed on the path 605. The training system may update this parameter to change the radius 603 for determining the spatial position of a training target. For example, the system may increase the radius 603 so that an orb appears farther from the user 610, or may decrease the radius 603 so that the orb appears closer to the user 610. In some embodiments, the system may select the radius 603 from a set of predefined radii. For example, the system may select a first radius for an initial trial, and may later select a second radius for a subsequent trial based on the assessed range of motion of the user 610 or one or more other training parameters determined for that user. By altering the radius 603 in this manner, the system may change the apparent depth of the training target, may change the portion of the augmented reality environment in which the training target appears, and may thereby alter the difficulty or character of the visual trial.

In some embodiments, one or more additional training parameters may be altered to change the difficulty of the visual trials for the user 610. For example, one training parameter may correspond to the size of a training target such as an orb. A smaller orb may require greater positioning accuracy and may therefore be suitable for a more advanced user. A larger orb may be easier to align with and may therefore be suitable for a less advanced user or for an earlier stage of training. Other training parameters may include movement speed, hold time, movement pattern, movement direction and range-related settings used to determine where and how a training target is presented. For example, a trial may be made more difficult by increasing target speed, decreasing target size, increasing hold time, selecting a direction of orb movement that is more challenging for the user, or selecting a more complex movement pattern, such as a sinusoidally varying path. For instance, where the user has greater pain, reduced mobility, or functional compromise on one side, such as difficulty tracking orbs moving to the right, the training system may increase presentation of orbs moving in that direction to provide additional challenge and training emphasis. A trial may be made less difficult by decreasing target speed, increasing target size, reducing hold time, or selecting a simpler path for the target. In this manner, the training system may alter one or more training parameters to tailor the visual trials to the assessed movement capability or performance of the user 610 over time.

At 406, the method may determine a start point and an end point for a training target. In some embodiments, the start point and the end point may be selected from within the range-of-motion limits of the user. The selected points may correspond to positions on the path 605. In some embodiments, a subsequent training target 620b may appear at any position on the path 605, provided that the selected position remains within the assessed movement region of the user.

At 408, the method may generate a path for movement of the training target. In some embodiments, the path may be generated between the selected start point and the selected end point. The generated path may include straight angular components, sinusoidally varying angular components, or a combination thereof. For example, an orb may move generally from left to right while also following a smooth wave-like deviation.

At 410, the method may select a target speed in visual trials where the training target is configured to travel along a path instead of appearing at a single spatial position. Target speed may be one training parameter used to affect difficulty of the visual trial. A slower speed may be used for an earlier trial or for a user having a more limited assessed range of motion. A greater speed may be used for a later trial or for a user having improved control.

At 412, the method may assess the range of motion of the user during the visual trial. In some embodiments, the display system may include one or more sensors that generate movement data indicative of position, orientation, or motion of the headset as the user moves toward the displayed training target. The system may evaluate an alignment between movement of the user and the spatial position of the training target. The system may determine whether the user reaches alignment. In some embodiments, the system may determine whether the user maintains alignment for a predetermined threshold time. For example, a crosshair fixed relative to the user's field of view may guide the user toward the orb. The system may determine whether the crosshair remains aligned with the orb for about one second.

At 414, the method may evaluate one or more performance metrics. In some embodiments, the performance metrics may include a smoothness metric corresponding to the assessed movement over a predetermined period of time. The system may determine whether movement toward the orb is steady or irregular. In some embodiments, the system may determine a baseline smoothness metric by aggregating smoothness metrics over a plurality of visual trials. The evaluated performance metrics may then be used to determine whether the current trial sequence should end at 416 or whether adaptive mode 420 should be used.

After evaluation of the one or more performance metrics at 414, the method may determine whether the current trial session should end at 416 or whether a subsequent trial should be initiated. In some embodiments, the method may end the trials when a selected trial session has been completed or when the system determines that no further trial adjustment is to be applied. If the trials are not ended, the method may proceed to 418 to determine training parameters for a subsequent trial. In some embodiments, the training parameters determined at 418 may be based on the assessed range of motion of the user and on one or more evaluated performance metrics from the earlier trial. For example, the system may determine one or more updated parameters for a subsequent orb-based trial, including a selected position, movement speed, target size, hold time, radius, or movement pattern for the subsequent training target.

At 422, the method may initiate a subsequent trial using the updated training parameters. In some embodiments, the subsequent trial may correspond to presentation of the subsequent training target 620b in FIG. 6A. The spatial position of the subsequent training target 620b may be based on the range of motion assessed during the earlier trial and on one or more updated parameters determined at 418. For example, the subsequent training target 620b may be positioned at another location along the path 605 that remains within the assessed movement region of the user 610. The user may then be guided toward the subsequent training target 620b after completion of the earlier trial. In some embodiments, the spatial position of the subsequent training target 620b may therefore be based on the assessed range of motion. For example, where the assessed range of motion indicates that the user 610 can comfortably rotate farther in one direction than another, the subsequent training target 620b may be positioned accordingly on the path 605.

At 424, the method may upload training parameters to a database 405. In some embodiments, the uploaded information may include user-specific movement limits, selected orb positions, selected radii, target speeds, target sizes, hold-related settings, movement patterns, performance metrics, or other session-related information. This information may be stored for later retrieval or use in a subsequent training session.

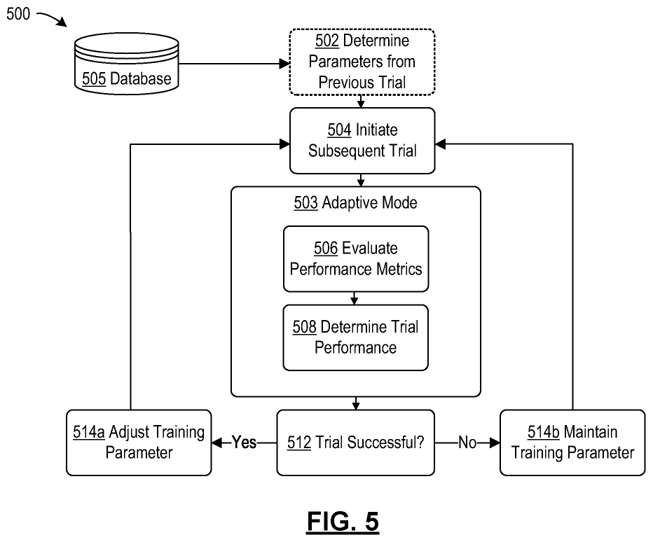

Reference is next made to FIG. 5 and FIG. 6B. FIG. 5 shows a flow diagram of an example method 500 in accordance with some embodiments. FIG. 6B illustrates an example visual trial 650 in which a training target moves along a path within an augmented reality environment. In some embodiments, when adaptive mode is active, the training system may determine whether a subsequent visual trial should increase a movement speed of the training target, decrease the movement speed of the training target, or keep the movement speed the same.

In some embodiments, the adaptive operation shown in FIG. 5 may correspond to determining training parameters based on tracked movement and one or more performance metrics. For example, tracked movement associated with a visual trial may define one or more performance metrics. The one or more performance metrics may include, for example, metrics derived from tracked movement of the user during the visual trial, such as, but not limited to, a smoothness metric, success indicator, or other metrics of user performance.

The system may evaluate one or more performance metrics associated with a visual trial to determine a trial performance. The trial performance may correspond to an evaluation of how well the user performed during a visual trial, as determined from the one or more performance metrics associated with that visual trial. For example, the trial performance may reflect whether the user was able to maintain alignment with the training target, how smoothly the user moved while tracking the training target, whether the user successfully tracked the training target for at least a threshold hold time while the training target was moving, and/or how consistently the user performed relative to one or more prior visual trials. In some embodiments, the system may determine the trial performance based on a single performance metric or based on a combination of performance metrics, such as a smoothness metric, a success indicator, or other tracked movement data. The determined trial performance may then be used to classify the user's performance during the visual trial as, for example, improved, reduced, successful, unsuccessful, more stable, less stable, or otherwise indicative of a level of training capability, thereby allowing the system to adjust one or more training parameters for a subsequent visual trial.

Based on the determined trial performance, the processor may adjust a training parameter of a subsequent training target for presentation in the subsequent visual trial. For example, the adjusted training parameter may include a movement speed, movement direction, target size, hold-related setting, movement pattern, or spatial position associated with the subsequent training target, such that the subsequent visual trial is adapted in accordance with the evaluated trial performance.

In some embodiments, the visual trial 650 shown in FIG. 6B may be defined relative to a coordinate system having axes 652, 654, and 656. The user 660 may generally remain centered within the augmented reality environment while moving to align his or her view with a displayed training target. In some embodiments, the user 660 may adjust yaw when the training target appears left or right, may adjust pitch when the training target appears above or below, and may exhibit roll as part of natural head movement while tracking the target. The environment may therefore define movement of the user in three dimensions while the user remains substantially in the same physical position.

FIG. 6B may further include a radius 653 and a path 655. In some embodiments, the radius 653 may define a distance between the user 660 and a displayed training target. The radius 653 may remain fixed for a given visual trial. In other embodiments, the radius 653 may be altered to place the training target farther from or closer to the user 660. The path 655 may define a line, arc, ring segment, spherical path, or other locus of points positioned at the selected radius 653 from the user 660. In this manner, a training target may move along the path 655 while remaining at a selected distance from the user 660.

In the example shown in FIG. 6B, a training target may begin at position 670. The training target may then move along the path 655 to an intermediate position 671 and may end at position 672. In some embodiments, the training target may be rendered as an orb. The user 660 may be guided to track the orb as it moves. For example, a crosshair fixed relative to the user's field of view may indicate the center of the user's view. The user may move his or her head and neck so that the crosshair remains aligned with the moving orb as the orb travels from 670 to 671 and then to 672.

At 502, the method may determine one or more parameters from a previous trial. In some embodiments, the one or more parameters may be retrieved from a database 505. The previous trial may be a trial such as the moving-orb trial shown in FIG. 6B. The parameters determined from the previous trial may include movement speed, path type, selected radius, target size, hold-related settings, or one or more performance metrics derived from tracked movement of the user.

At 504, the method may initiate a subsequent trial using those parameters. In some embodiments, the subsequent trial may again present an orb within the augmented reality environment and may cause the orb to move along a selected path at a selected speed.

At 506, the method may evaluate one or more performance metrics as part of an adaptive mode 503. In some embodiments, the performance metrics may be based on tracked movement of the user while the user follows the moving training target.

In some examples, the performance metrics may include a smoothness metric for the visual trial. The system may also determine a baseline smoothness metric over a plurality of visual trials. The baseline smoothness metric may be determined by aggregating smoothness metrics over a plurality of visual trials. For example, the system may calculate an average smoothness value over the last five trials. In another example, the system may calculate a weighted average that gives greater weight to more recent trials. In a further example, the baseline smoothness metric may be calculated as a median value, a moving average, or another representative measure derived from previously stored smoothness values. The baseline smoothness metric may therefore represent a reference level for the user's expected tracking performance. The system may then compare the smoothness metric to the baseline smoothness metric and use the difference to determine whether speed of a subsequent training target is to be increased, decreased, or maintained.

In some embodiments, the system may additionally use the comparison between the smoothness metric and the baseline smoothness metric to determine whether a direction of movement for a subsequent training target is to be changed. For example, where the performance metrics indicate that movement in a particular direction is more difficult for the user, the system may adjust the subsequent visual trial so that the training target moves in a different direction or more frequently in the identified direction to provide adaptive training responsive to the user's performance.

In other examples, the one or more performance metrics may additionally or alternatively include a success indicator associated with the visual trial. The success indicator may indicate whether the user successfully tracked the training target for at least a threshold hold time while the training target was moving. The threshold hold time may correspond to a minimum duration for which the user is to maintain tracking of the training target while the training target is moving in order for the visual trial to be identified as successful. The threshold hold time may be predetermined, may be selected by a clinician, or may be dynamically adjusted by the system responsive to the user's performance. For example, the threshold hold time may be between about 0.5 seconds and about 3 seconds, and in one example may be about 1 second. Where the user maintains tracking of the moving training target for at least the threshold hold time, the system may determine that the visual trial was successfully completed. Where the user fails to maintain tracking of the moving training target for at least the threshold hold time, the system may determine that the visual trial was unsuccessful.

At 508, the method may determine a trial performance. The trial performance may correspond to an overall assessment of how well the user performed during the visual trial based on the one or more performance metrics evaluated at 506. For example, the trial performance may indicate whether the user's tracking performance during the visual trial was improved, reduced, successful, unsuccessful, stable, unstable, or otherwise representative of the user's ability to follow the training target. The trial performance may also be determined from a single evaluated performance metric. For example, the trial performance may be based on the smoothness metric alone, such as by comparing the smoothness metric for the visual trial to a baseline smoothness metric determined from a plurality of prior visual trials. Where the smoothness metric is greater than or sufficiently close to the baseline smoothness metric, the method may determine that the trial performance is satisfactory or improved. Where the smoothness metric is less than the baseline smoothness metric by at least a threshold amount, the method may determine that the trial performance is reduced.

The trial performance may alternatively be determined from the success indicator alone. For example, where the success indicator indicates that the user successfully tracked the training target for at least the threshold hold time while the training target was moving, the method may determine that the trial performance is successful. Where the success indicator indicates that the user did not maintain tracking for at least the threshold hold time, the method may determine that the trial performance is unsuccessful. In this manner, the success indicator may provide a binary or categorical measure of whether the user completed the visual trial in accordance with a defined success criterion.

The trial performance may be determined based on a combination of two or more evaluated performance metrics. For example, the method may evaluate both the smoothness metric and the success indicator to determine whether the user not only completed the visual trial successfully, but also did so with a desired level of movement quality. In one example, where the success indicator shows that the user maintained tracking of the moving training target for at least the threshold hold time and the smoothness metric is at or above the baseline smoothness metric, the method may determine that the trial performance is strong or improved. In another example, where the user successfully maintained tracking for the threshold hold time but the smoothness metric is below the baseline smoothness metric, the method may determine that the trial performance is successful but less controlled, thereby indicating that the user completed the task but with reduced movement quality. In a further example, where the user failed to maintain tracking for the threshold hold time and the smoothness metric is also below the baseline smoothness metric, the method may determine that the trial performance is poor or reduced.

At 512, the method may determine whether the visual trial was successful based on the trial performance determined at 508. The trial performance may provide an assessment of how well the user performed during the visual trial based on one or more evaluated performance metrics. The method may use the trial performance to determine whether the visual trial satisfied one or more success criteria. For example, where the trial performance indicates that the user performed the visual trial at or above a threshold level, the method may determine that the visual trial was successful. Where the trial performance indicates that the user did not satisfy the threshold level, the method may determine that the visual trial was unsuccessful. In some embodiments, the determination at 512 may therefore be based on a single evaluated performance metric or on a combination of evaluated performance metrics reflected in the trial performance.

At 514, the method may adjust a training parameter 514a, or may maintain a training parameter 514b, based on whether the visual trial was determined to be successful at 512. In some embodiments, where the visual trial is determined to be successful, the method may increase or otherwise modify a training parameter for a subsequent visual trial to provide a greater training challenge. For example, the method may increase movement speed, adjust movement direction, reduce target size, select a more complex movement pattern, or otherwise alter presentation of a subsequent training target. In some embodiments, where the visual trial is determined to be unsuccessful, the method may decrease a training parameter, select a less challenging movement direction or movement pattern, or otherwise modify the subsequent visual trial to better correspond to the user's current capability. The method may maintain the training parameter substantially unchanged where the visual trial satisfies one criterion but not another, such as where the user successfully tracked the training target for the threshold hold time but the smoothness metric indicates that movement quality remains near a baseline level. In this manner, the determination at 512 may be used to decide whether a subsequent visual trial is to be made more difficult, less difficult, or substantially unchanged.

It will be understood, however, that other adaptive modes may also be used. In some embodiments, another adaptive mode may adjust target size, hold time, selected radius, selected path, start position, end position, or movement pattern for a subsequent visual trial. For example, a smaller target, a longer hold time, a greater movement range, or a more complex trajectory may be selected to increase difficulty. A larger target, a shorter hold time, a reduced movement range, or a simpler trajectory may be selected to reduce difficulty. In this manner, the training system may implement different adaptive modes to adjust different training parameters based on assessed range of motion, tracked movement, or one or more performance metrics determined from one or more prior visual trials.

In some embodiments, two or more adaptive modes may be used in parallel. For example, the training system may simultaneously adjust movement speed, movement direction, target size, hold time, selected radius, selected path, or movement pattern for a subsequent visual trial. In some embodiments, these parameters may be updated together based on the assessed range of motion of the user, tracked movement of the user, or one or more performance metrics determined from one or more prior visual trials. For example, where prior performance indicates improved control, the training system may increase target speed while also decreasing target size or selecting a more complex movement pattern. Conversely, where prior performance indicates that a less demanding trial is appropriate, the training system may decrease target speed while also increasing target size or selecting a simpler path. In this manner, multiple adaptive modes may operate concurrently to tailor the subsequent visual trial to the user.

The moving training target of FIG. 6B may follow different trajectories. The training system may select a start position and an end position for the training target from within range-of-motion limits of the user. The system may then generate a trajectory between those positions. In some embodiments, the trajectory may include straight angular components, sinusoidally varying angular components, or a combination thereof. For example, a simpler trial may use a substantially linear trajectory from 670 to 672. A more difficult trial may use a sinusoidally varying path in which the orb moves generally toward the end position while also following a wave-like deviation. In some embodiments, diagonal paths, curved paths, or compound paths may also be used to vary difficulty.

The selected trajectory may be used together with selected speed to affect difficulty of the visual trial. For example, a slower orb moving along a substantially straight path may be suitable for an earlier or easier trial. A faster orb moving along a sinusoidally varying path may be suitable for a later or more advanced trial. In some embodiments, the system may also vary one or more additional parameters, such as target size, hold time, or radius, in combination with speed and trajectory. For example, a smaller orb may require greater positioning accuracy whereas a larger orb may be easier to track. Similarly, a greater radius may alter apparent depth, whereas a smaller radius may place the orb closer to the user, making it easier to follow.

In some instances, the system may determine that the user has moved off course while tracking the training target. For example, the system may determine that a crosshair or other visual indicator no longer remains aligned with the moving orb, or may determine that deviation between the user's view and the target path exceeds a threshold. In response, the system may continue the trial and allow the user to realign with the orb or the system may pause or slow movement of the orb. Alternatively, the system may stop the orb at its current position or may reset a hold timer. The system may restart the trial or initiate another trial with reduced speed or a simpler path. In some embodiments, the system may record the off-course event as part of the performance metrics used for later adaptive decisions.

In some embodiments, after the adaptive decision of FIG. 5 is made, the selected training parameters may be uploaded to the database 505. The uploaded information may include a current smoothness metric, a baseline smoothness metric, the calculated difference, the selected speed adjustment decision, the selected path, and one or more other trial settings. This information may be stored for later retrieval or use in another subsequent visual trial. In this manner, the method of FIG. 5 may provide an adaptive framework for changing speed of a moving training target in response to tracked performance during the type of visual trial illustrated in FIG. 6B.

In some embodiments, where the system is deployed in a clinical setting, the system enables a clinician or assistant to supervise multiple patients concurrently, as the training sessions may be performed semi-autonomously following initial setup. In some embodiments, performance data can be recorded and transmitted to a clinician-facing interface, where longitudinal metrics such as improvements in range of motion, movement smoothness, and functional task scores can be visualized. This enables objective tracking of user progress.

In another embodiment, the system can be deployed for at-home use, wherein the user is provided with the augmented reality device and a dedicated computing unit such as a mobile phone or a personal computer. The system operates in a remote mode, allowing patients to complete prescribed training sessions independently while maintaining connectivity with a clinician dashboard. The clinician is able to remotely monitor adherence, review performance metrics, and modify training protocols as needed. The adaptive algorithm may further personalize the training program by incrementally adjusting task parameters based on session performance data, ensuring progression is tailored to the user's capabilities.

For example, a user presenting with reduced cervical rotation and impaired movement control may begin with low-speed, linear target tracking tasks within a restricted range of motion. Over successive sessions, the system may increase task complexity by expanding range of motion limits, introducing multi-directional or sinusoidal trajectories, and increasing movement speed, contingent on demonstrated improvements in performance metrics. Corresponding data visualizations may illustrate progressive increases in ROM (e.g., from 45° to 70° head rotation), reductions in movement variability, and improvements in task accuracy over time.

In some implementations, aggregated user data may demonstrate clinically meaningful outcomes, such as improvements in functional disability, and increased adherence to prescribed training protocols. Combining in-clinic use with remotely monitored at-home training, enables continuous training, enhances patient engagement, and provides clinicians with objective training data.

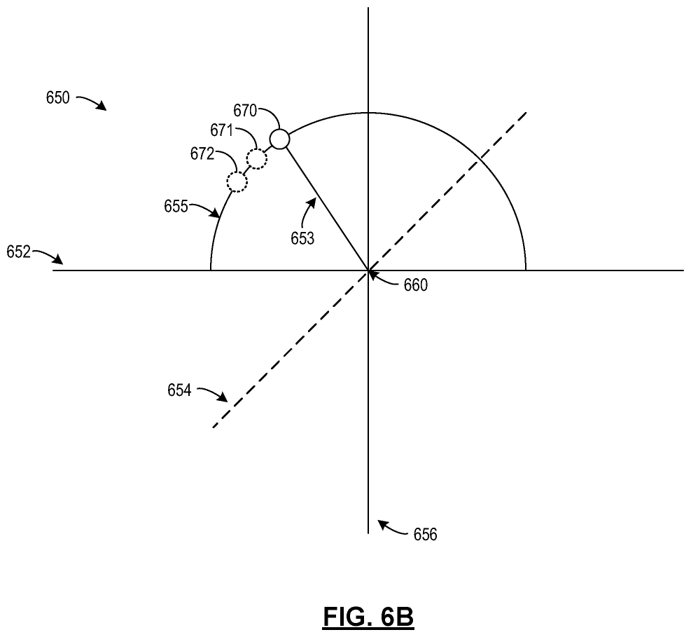

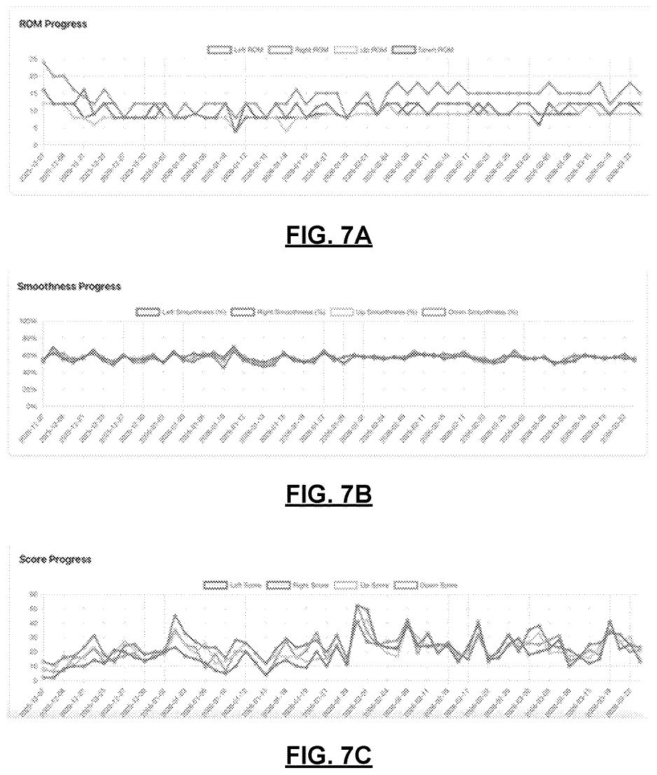

Reference is next made to FIG. 7A to FIG. 7C, which shows progress of a patient over several training sessions using the system as described herein. Over several weeks of training with the system, improvements for neck movements in leftward, rightward, upward, and downward movement are observed. Specifically, improvements in range of motion function, as shown in FIG. 7A, and improvements in smoothness of movements, as shown in FIG. 7B. In addition, a performance score, corresponding to how successfully the user was able to complete the tasks in each session was tracked over time, as shown in FIG. 7C. This training enables continuity of care outside the of clinic, allowing users to engage in neck training more frequently while also increasing access to neck training.

The device tracks the user's performance during remote sessions, thereby allowing clinicians to monitor progression, maintenance, or regression of the user's neck function and mobility over time. This provides critical insights into patient progress and supports more informed assessment and management for the clinician.

While the above description describes features of example embodiments, it will be appreciated that some features and/or functions of the described embodiments are susceptible to modification without departing from the spirit and principles of operation of the described embodiments. For example, the various characteristics which are described by means of the represented embodiments or examples may be selectively combined with each other. Accordingly, what has been described above is intended to be illustrative of the claimed concept and non-limiting. It will be understood by persons skilled in the art that other variants and modifications may be made without departing from the scope of the invention as defined in the claims appended hereto. The scope of the claims should not be limited by the preferred embodiments and examples, but should be given the broadest interpretation consistent with the description as a whole.

1. A training system for determining spatial positions of training targets for visual trials presented within an augmented reality environment, the training system comprising:
a display system configured to present the visual trials within the augmented reality environment; and
one or more sensors configured to track movement of a user;
a processor, the processor configured to:
generate a visual trial presented to the user via the display system, the visual trial having at least one training target presented at a spatial position within the augmented reality environment;
obtain, from the one or more sensors of the display system during the visual trial, the movement of the user;
assess a range of motion of the user by evaluating an alignment between the movement of the user and the spatial position of the training target during the visual trial to determine training parameters for a subsequent visual trial defining a subsequent spatial position for a subsequent training target;
generate the subsequent visual trial presented to the user via the display system, the subsequent visual trial having the subsequent training target; and
apply the training parameters to the display system presenting the subsequent visual trial to determine the subsequent spatial position of the subsequent training target within the augmented reality environment.

2. The training system of claim 1, wherein the processor is configured to determine the spatial position for the subsequent training target by selecting a start position and an end position for the subsequent training target from within range of motion limits of the user.

3. The training system of claim 2, wherein the processor is configured to generate a trajectory for the training target between the start position and the end position along a path generated from a combination of straight angular components and sinusoidally varying angular components.

4. The training system of claim 1, wherein, to assess the range of motion of the user, the processor is configured to determine whether movement of the user is substantially aligned with the training target for a predetermined threshold time.

5. The training system of claim 1, wherein the processor is configured to determine the training parameters for the subsequent visual trial based on tracked movement associated with the visual trial, the tracked movement defining one or more performance metrics.

6. The training system of claim 5, wherein the one or more performance metrics comprise a smoothness metric for the visual trial based on the movement, the smoothness metric corresponding to the range of motion assessed over a predetermined period of time.

7. The training system of claim 5, wherein the one or more performance metrics comprise a success indicator associated with the visual trial, the success indicator indicating whether the user successfully tracked the training target for at least a threshold hold time while the training target was moving.

8. The training system of claim 5, wherein, to determine the training parameters for the subsequent visual trial, the processor is configured to:
evaluate the one or more performance metrics to determine a trial performance; and
adjust a training parameter of the subsequent training target in the subsequent visual trial based on the trial performance.

9. The training system of claim 1, wherein the processor is configured to calibrate the range of motion of the user by iteratively presenting training targets along an axis and recording positional limits for the movement.

10. The training system of claim 1, wherein the processor is configured to store the training parameters in a remote database upon completion of the visual trial.

11. A computer-implemented method for determining spatial positions of training targets for visual trials presented within an augmented reality environment, the method comprising:
generating a visual trial presented to a user via a display system, the display system having sensors configured to track movement of the user, the visual trial having at least one training target presented at a spatial position within the augmented reality environment;
obtaining, from the sensors of the display system during the visual trial, the movement of the user;
assessing a range of motion of the user by evaluating an alignment between the movement of the user and the spatial position of the training target during the visual trial to determine training parameters based on the range of motion of the user for a subsequent visual trial defining a subsequent spatial position for a subsequent training target;
generating the subsequent visual trial presented to the user via the display system, the subsequent visual trial having the subsequent training target;
and applying the training parameters to the display system presenting the subsequent visual trial to determine the subsequent spatial position of the subsequent training target within the augmented reality environment.

12. The method of claim 11, further comprising determining the spatial position for the subsequent training target by selecting a start position and an end position for the subsequent training target from within the range of motion limits.

13. The method of claim 12, further comprising generating a trajectory for the training target between the start position and the end position along a path generated from a combination of straight angular components and sinusoidally varying angular components.

14. The method of claim 11, wherein assessing the range of motion of the user further comprises determining whether the movement is substantially aligned with the training target for a predetermined threshold time.

15. The method of claim 11, wherein the training parameters are determined based on tracked movement associated with the visual trial, the tracked movement defining one or more performance metrics.

16. The method of claim 15, wherein the one or more performance metrics comprise a smoothness metric for the visual trial based on the movement, the smoothness metric corresponding to the range of motion assessed over a predetermined period of time.

17. The method of claim 15, wherein the one or more performance metrics comprise a success indicator associated with the visual trial, the success indicator indicating whether the user successfully tracked the training target for at least a threshold hold time while the training target was moving.

18. The method of claim 15, wherein determining training parameters for the subsequent visual trial comprises:
evaluating the one or more performance metrics to determine a trial performance; and
adjusting a training parameter of the subsequent training target in the subsequent visual trial based on the trial performance.

19. The method of claim 11, further comprising calibrating the range of motion of the user by iteratively presenting training targets along an axis and recording positional limits for the movement.

20. The method of claim 11, further comprising storing the training parameters in a remote database upon completion of the visual trial.
